(19) United States
(12) Patent Application Publication
(10) Pub. No.: US 2021/0219863 A1
(43) Pub. Date: Jul. 22, 2021

(54) Electrical Impedance Measurement and EIT Image for Location of a Micro Bio-Channel Under Skin

(71) Applicant: Wei WANG, Oadby, Leicester Leicestershire (GB)

(72) Inventors: Wei WANG, (US); Weibo ZHANG, Beijing (CN)

(21) Appl. No.: 16/305,507

(22) PCT Filed: May 26, 2017
(86) PCT No.: PCT/GB2017/051514

§ 371 Date: Nov. 29, 2018

(30) Foreign Application Priority Data
May 31, 2016 (GB) 1609551.5

(51) Int. Cl. A61B 5/0532; A61B 5/00

(52) U.S. Cl. CPC A61B 5/0532; A61B 8/4416; A61B 2562/046; A61B 5/7235

(57) ABSTRACT

A method for locating a variation in one or more electrical impedance properties of an object, the method comprising the steps of: (i) obtaining electrical impedance data for the object at different locations; (ii) analysing the obtained electrical impedance data using a transfer function of an assumed electrical model to determine a variation of a plurality of electrical impedance properties for the object with location; and (iii) identifying a location identified by a variation of one or more of the plurality of electrical impedance properties.

### FIELD OF THE INVENTION

Embodiments of the present invention relate to electrical impedance measurement for location of a micro bio-channel under skin.

### BACKGROUND TO THE INVENTION

Electrical impedance tomography (EIT) is a known imaging technique, particularly used in medical and other applications for the detection of underlying abnormal morphology such as breast cancer. Typically, a plurality of electrodes is attached to an object to be imaged. Either input voltages are applied across a subset of ‘input’ electrodes and output electric currents are measured at ‘output’ electrodes, or input electric currents are applied between a subset of ‘input’ electrodes and output voltages are measured at ‘output’ electrodes or between pairs of output electrodes. For example, when a very small alternating electric current is applied between a subset of ‘input’ electrodes, the potential difference between output electrodes or between pairs of ‘output’ electrodes is measured. The current is then applied between a different subset of ‘input’ electrodes and the potential difference between the output electrodes or between pairs of ‘output’ electrodes is measured. An electrical impedance image based on variations in electrical impedance can then be constructed using an appropriate image reconstruction technique.

### BRIEF DESCRIPTION OF THE INVENTION

According to some embodiments of the invention there is provided a method for locating a variation in one or more electrical impedance properties of an object, the method comprising the steps of:

(i) obtaining electrical impedance data for the object at different locations;

(ii) analysing the obtained electrical impedance data using a transfer function of an assumed electrical model to determine a variation of a plurality of electrical impedance properties for the object with location; and

(iii) identifying a location identified by a variation of one or more of the plurality of electrical impedance properties.

Electrical impedance properties, which are related to the measured electrical impedance data, can be derived from the measured electrical impedance data, and these electrical impedance properties can be used to locate human morphology, such as for example a micro bio-channel under skin and/or a sub-cutaneous micro-volume of low hydraulic resistance and/or meridian and/or acupuncture point.

According to some embodiments of the invention there is provided a method for locating a variation in one or more parametric impedance values of an object, the method comprising the steps of:

(i) obtaining electrical impedance data for the object over a range of frequencies at different locations;

(ii) analysing the obtained electrical impedance data using a transfer function of an assumed electrical model to determine a variation of a plurality of electrical impedance properties for the object with location;

(iii) constructively combining selected ones of the determined plurality of electrical impedance properties to provide at least one parametric impedance value for the object that varies with location; and

(iii) identifying a location identified by a spatial variation of the at least one parametric impedance value for the object.

The electrical model may assume first and second serially connected impedances connected in parallel with a third impedance. The electrical model may assume a capacitor and a serially connected resistor, which are connected in parallel with another resistor. The electrical model may be a fractal model usable at any resolution.

The electrical impedance properties may be selected from the group comprising:

an impedance at a lower frequency limit,

an impedance at an upper frequency limit,

a relaxation frequency f₁ at which there is a change in the impedance,

an impedance at that relaxation frequency, and

the impedance gradient at that relaxation frequency.

The electrical model may assume a capacitance and a serially connected resistance, which are connected in parallel with a parallel resistance to form a model circuit having a relaxation frequency, wherein the parametric impedance value used for imaging is a combination of two or more of: the capacitance, the relaxation frequency, the serial resistance and the parallel resistance.

The electrical model may assume a ‘membrane’ capacitance and a serially connected intracellular resistance, which are connected in parallel with an extracellular resistance, wherein the parametric impedance value includes one of: Membrane impedance, membrane conductivity, Intracellular impedance product, Intracellular impedance difference, Intracellular impedance normalized difference, Intracellular impedance differential, Intracellular impedance normalized differential, Intracellular conductivity product, Intracellular conductivity difference, Intracellular conductivity normalized difference, Intracellular conductivity differential, Intracellular conductivity normalized differential, Intracellular time constant, Intracellular frequency constant, Extracellular impedance product, Extracellular impedance difference, Extracellular impedance normalized difference, Extracellular impedance differential, Extracellular impedance normalized differential, Extracellular conductivity product, Extracellular conductivity difference, Extracellular conductivity normalized difference, Extracellular conductivity differential, Extracellular conductivity normalized differential, Extracellular time constant Extracellular frequency constant, Extra-intra impedance product, Extra-intra impedance difference, Extra-intra impedance normalized difference, Extra-intra differential Extra-intra normalized differential, Extra-intra conductivity product, Extra-intra conductivity difference, Extra-intra conductivity normalized difference, Extra-intra conductivity differential, Extra-intra conductivity normalized differential, any one of the preceding parameters modified by a dispersion gradient α.

The electrical model may assume a first impedance and a serially connected second impedance, which are connected in parallel with a third impedance to form a model circuit having a relaxation frequency, wherein the parametric impedance value is a combination of two or more of: the first impedance, the relaxation frequency, the second impedance and the third impedance.

The electrical model may assume an inclusion boundary impedance and a serially connected intra-inclusion impedance, which are connected in parallel with an inter-inclusion impedance, wherein the parametric impedance value includes one of: inclusion boundary impedance, inclusion boundary conductivity, Intra-inclusion impedance product Intra-inclusion impedance difference, Intra-inclusion impedance normalized difference Intra-inclusion impedance differential, Intra-inclusion impedance normalized differential Intra-inclusion conductivity product, Intra-inclusion conductivity difference, Intra-inclusion conductivity normalized difference, Infra-inclusion conductivity differential, Infra-inclusion conductivity normalized differential, Intra-inclusion time constant, Intra-inclusion frequency constant, Inter-inclusion impedance product, Inter-inclusion impedance difference, Inter-inclusion impedance normalized difference, Inter-inclusion impedance differential, Inter-inclusion impedance normalized differential, Inter-inclusion conductivity product, Inter-inclusion conductivity difference, Inter-inclusion conductivity normalized difference Inter-inclusion conductivity differential, Inter-inclusion conductivity normalized differential Inter-inclusion time constant, Inter-inclusion frequency constant, Inter-intra impedance product, Inter-intra impedance difference, Inter-intra impedance normalized difference Inter-intra differential, Inter-intra normalized differential, Inter-intra conductivity product Inter-intra conductivity difference, Inter-intra conductivity normalized difference Inter-intra conductivity differential, Inter-intra conductivity normalized differential any one of the preceding parameters modified by a dispersion gradient α.

The frequency range may be between 0 and 20 MHz. The frequency range may be between 0 and 100 MHz.

According to some embodiments of the invention there is provided a method for locating a variation in parametric impedance values of an object, the method comprising the steps of:

(i) obtaining electrical impedance data for the object at different locations;

(ii) analysing the obtained electrical impedance data to determine a variation of a plurality of electrical impedance properties for the object with location;

(iii) constructively combining selected electrical impedance properties from said plurality of electrical impedance properties to provide parametric impedance values for the object that vary with location;

(iv) identifying a location identified by a spatial variation of one or more of the parametric impedance values for the object.

The electrical impedance data for the object may be collected with a frequency bandwidth of between 0 and 20 MHz-100 MHz for biological materials.

Step (iii) may comprise combining predetermined electrical impedance properties according to an impedance emphasising algorithm.

Step (i) may comprise obtaining electrical impedance data for the object at a plurality of frequencies. For biological materials the transfer function may be given by the Cole-Cole formula [Cole, 1920; Cole, 1924] over the frequency range 0-100 MHz.

The method may be used to analyse an electrically conductive object having a cellular structure or cell-like structure, and step (ii) may comprise the use of an equivalent electrical impedance circuit to model the structure, such as Cole-Cole model [Cole, 1920; Cole, 1924]

The equivalent electrical impedance circuit may in the limiting case comprise a cell membrane capacitance (C), an intracellular resistance (Rᵢ), and an extracellular resistance (Rₑ). Wherein the equivalent electrical impedance circuit comprises the cell membrane capacitance (C) in series with the intracellular resistance (Rᵢ), the cell membrane capacitance (C) and intracellular resistance (Rᵢ) being in parallel with the extracellular resistance (Rₑ) or an equivalent electrical circuit.

The electrical impedance properties may be selected from the group consisting of Rᵢ (cell/Group intra resistance), Rₑ (Cell/Group extra resistance), C (Cell/Group capacitance), fᵣ (cell/group relaxation frequency) and a (cell/group relaxation factor).

Step (iii) may comprise combining fᵣ (relaxation frequency) and C (cell/group capacitance) by multiplication which may provide parametric impedance values.

An ultrasound transducer may perform ultrasound detection by applying a first ultrasound signal to the body tissue, receiving an ultrasound response signal characteristic of the body tissue, and providing a second output signal representative of the ultrasound response signal,

The ultrasound transducer and the electrode array for obtaining electrical impedance data may be mounted on a movable element of the apparatus

A spacing member may intervene between the movable element and the subject.

The spacing member may comprise one or more apertures. The apertures of the spacing member may be configured for alignment, in use, with electrodes of the electrode array. The spacing member may be ultrasonically transparent. The spacing member may be non-conductive.

The rotatable element may have a window (e.g. an aperture in the rotatable element.) for the ultrasound transducer. The electrodes of the electrode array may be supported over the window or may not be present over the window.

According to some embodiments of the invention there is provided a computer program providing instructions for a processor to perform any of the above defined methods.

According to some embodiments of the invention there is provided a system or apparatus comprising means for performing any of the above defined methods.

### BRIEF DESCRIPTION OF THE DRAWINGS

For a better understanding of the present invention reference will now be made by way of example only to the accompanying drawings in which:

FIG. 1 is a diagrammatic illustration of electrical impedance measurement apparatus;

FIGS. 2A and 2B show graphs of measured electrical impedance as a function of frequency with single or multiple dispersion;

FIG. 3 shows an example electrical impedance circuit model of an object having a cellular or cellular-like structure at the “micro-scale”;

FIG. 4 shows a generic electrical impedance circuit model of an object having a cellular or cellular-like structure at a “macro-scale”;

FIG. 5 is a diagrammatic illustration of an electrical impedance measurement apparatus similar to that illustrated in FIG. 1 but comprising an ultrasound detector;

FIGS. 6A, 6B, 6C, 6D illustrated different examples of electrode arrays;

FIG. 7 is an example of an electrical impedance measurement apparatus for locating a micro bio-channel under skin, a meridian or an acupuncture point; and

FIG. 8 is an example of an electrical impedance measurement apparatus for locating a micro bio-channel under skin, a meridian or an acupuncture point.

### DETAILED DESCRIPTION OF EMBODIMENTS OF THE INVENTION

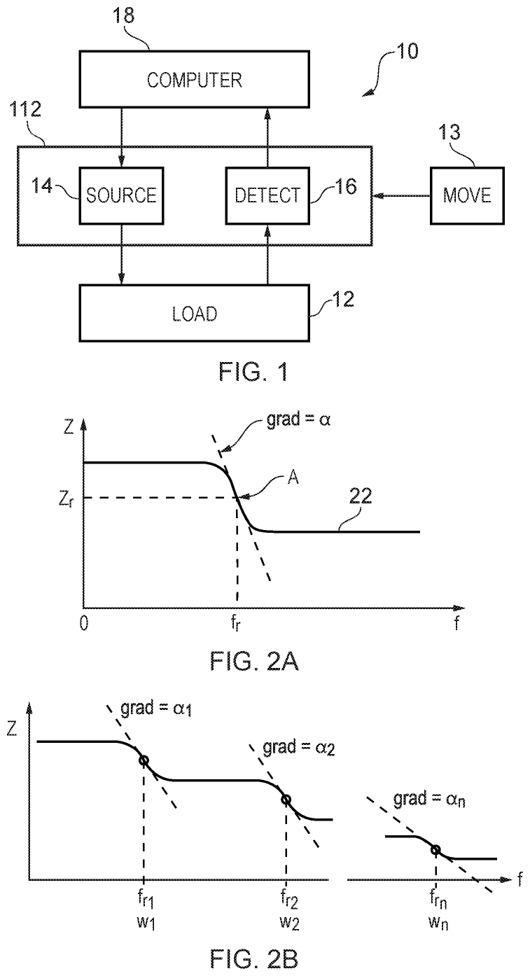

FIG. 1 illustrates diagrammatically electrical impedance measurement or electrical impedance tomography (EIT) apparatus 10 for measuring impedance data for a load 12. The load 12 comprises an electrically conductive object to which are attached a plurality of electrodes. The term ‘electrically conductive’ means that the object is capable of conducting an electric current but it does not necessarily need to conduct current very well. The object may be a living animal referred to as a subject, for example, a human referred to as a human subject.

The apparatus 10 further comprises a signal controller 112 comprising a signal source 14 and a signal detector 16 and a computer 18. In one embodiment, the signal source provides, as an input signal, an electric current and the signal detector detects, as an output signal, voltage. In another embodiment, the signal source provides, as an input signal, a voltage and the signal detector detects, as an output signal, electric current.

The computer 18 typically comprises at least a processor and a memory. The memory stores a computer program which when loaded into the processor controls the computer.

The input signal is applied using the source 14 to the object via electrodes and the resulting output signals present at same or other electrodes are measured using the detector 16. This process is repeated for different frequencies of input signal. For example, the electric signal may be applied by the signal source 14 at a number of frequencies between 0 Hz (direct current) and 20 MHz or 100 MHz, to enable frequency dependent electrical impedance data to be obtained for the object.

The separation of the electrodes used for the impedance measurements determines the resolution or scale at which the object is analysed. The electrical impedance measurements may be obtained at an expected scale of interest (e.g. micro-meter or millimetre range). As an example of the scale of interest, for a biological object, we may be interested in the single cell or in the group cell level or at tissue or histology level.

Subsequently the obtained electrical impedance data will be analysed using a transfer function of an assumed electrical model to determine a plurality of electrical impedance properties for the object. The electrical model used may depend upon the resolution/scale of the impedance measurements.

The apparatus 10 also comprises a system 13 for moving the electrodes used by controller 112 to a different location relative to the load 12. The movement may be a physical movement that physically translates the electrodes to a different physical location or may comprise reconfiguring the electrodes used by the electrode array so that there is an effective movement of the electrode array.

The plurality of electrical impedance properties for the object are determined at a location then the location is changed and the plurality of electrical impedance properties for the object are determined again. Therefore the plurality of electrical impedance properties for the object may be automatically determined at each one of multiple different locations.

The apparatus 10 then identifies a location from a spatial variation of one or more of the plurality of electrical impedance properties. For example, a micro bio-channel under skin, a meridian or acupuncture point is identified by a predetermined spatial variation of one or more of the plurality of electrical impedance properties.

Referring to FIGS. 2A and 2B, the electrical impedance data obtained using the above method can be plotted as a function of frequency. This plot 22 represents the impedance changes vs frequencies or transfer function for the object. The computer 18 is operable to execute an appropriate algorithm to analyse the obtained impedance transfer function or frequency dependent impedance properties and thereby determine a plurality of electrical impedance properties for the object at different locations.

The electrical impedance properties typically include one or more of:

a) the impedance at the limit ω->0 (lower limit)

b) the impedance at the limit ω->00 (upper limit)

c) (i) the relaxation frequency at which there is a change in the impedance
(ii) the impedance at that change frequency
(iii) the gradient of the change of impedance, particularly at the relaxation frequencies;

For example, if there are N dispersions including the Alpha, Beta and Gamma dispersions of biological materials [Cole K S, Permeability and impermeability of cell membranes for ions. Cold Spring Harbor Symp. Quant. Biol. 8 pp 110-22, 1940] within the frequency range used, where N>1, then the dispersion frequencies ω₁, ω₂, . . . ω_{N-1}, ω_{N}, are identified and the electrical impedance properties for a particular dispersion m would typically include one or more of:

a) For m=1, the impedance at the lower (global) limit ω->0
For m>1, the impedance at the lower (local) limit ω->ωₘ−a, where a<(ωₘ−ωₘ₋₁) and may possibly be %(ωₘ−ωₘ₋₁)

b) For m=N, the impedance at the upper (global) limit ω->∞
For m<N, the impedance at the upper (local) limit ω->ωₘ+b, where b<(ωₘ₊₁−ωₘ) and may possibly be b ˜½(ωₘ₊₁−ωₘ)

c) (i) the relaxation frequency ωₘ (fᵣₘ) at which there is a change in the impedance
(ii) the impedance at that change frequency
(iii) the gradient of the change

The amount of variation of one or more of these impedance properties can be used to analyse the structure of the object due to the intra/extra cellular or intra/extra cellular-like related changes.

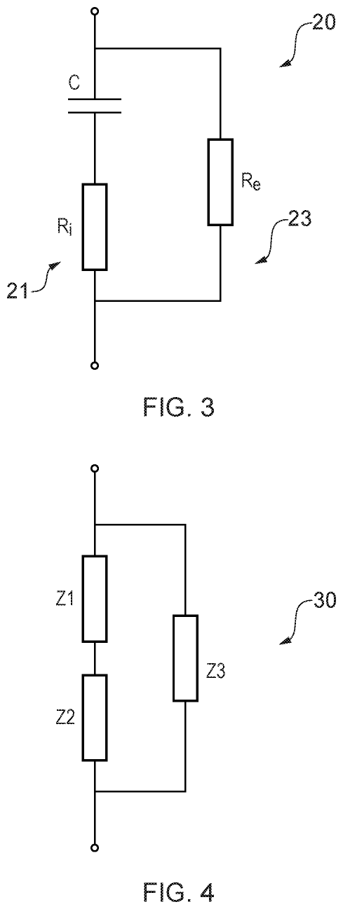

In some embodiments, the object under analysis is modeled using an equivalent electrical impedance circuit. The object may be modeled using an equivalent electrical impedance circuit 20 illustrated in FIG. 3. Objects which may be modeled using the equivalent electrical impedance circuit 20 may, include human or animal tissue.

In the illustrated embodiment, the equivalent electrical impedance circuit 20 comprises a cell portion 21 in parallel with an extra-cell portion 23. The cell portion 21 has a capacitance C and a resistance Rᵢ in series. The resistance C is associated with the cell membrane/boundary and the resistance Rᵢ, is associated with the interior of the cell. The extra-cell portion 23 has a resistance Rₑ. The resistance Rₑ is associated with the structure outside the cell. The resistance Rₑ is connected in parallel with the series connected capacitance C and resistance Rᵢ.

A non-limiting example of a single dispersion impedance transfer function for this circuit is:

$$Z ( ω ) = \frac{Re ( 1 + j \cdot ω \cdot C \cdot Ri )}{1 + j \cdot ω \cdot C \cdot ( Re + Ri )}$$

In the limit ω->0, Z->Rₑ

In the limit ω->∞, Z->Rᵢ//Rₑ i.e. Rᵢ Rₑ/(Rᵢ+Rₑ)

There is a change (dispersion) at frequency fr and an impedance Zr that has a gradient α.

The transfer model for multiple dispersion in biological tissue can be modeled by the Cole-Cole equation (Cole K S 1940, Cole K S 1941, McAdams E T et al, 1995) as follows:

Z=R∝+(R0−R∝)/(1+(jf/fr))(1−α)

Usually this equation can be rewritten as the equation below if a three-element electrical equivalent circuit is used for a simple modeling cell suspensions (Fricke and Morse, 1925) or tissues:

Z=Rₑ·Rᵢ/(Rₑ+Rᵢ)+(Rₑ−Rₑ·Rᵢ/(Rₑ+Rᵢ))/(1+(jf/fr))(1−α)

Where R∝ is the result of paralleling Rₑ and Rᵢ.

There are changes (dispersion) at frequency fᵣᵢ and impedance Zᵣᵢ that has a gradient αᵢ.

As indicated above, the computer 18 is operable to execute an appropriate algorithm to analyse the measured impedance data and extract a plurality of electrical impedance properties for the object under analysis at each location. For example, based on the measured impedance data, the algorithm may be operable to plot impedance data points as a function of frequency and produce a best fit line 22 using the model to form the transfer function illustrated in FIG. 2. From this transfer function, the computer 18 is capable of determining a plurality of individual impedance properties for the object. These impedance properties may include:

a) the impedance at the limit ω->0, which gives Rₑ

b) the impedance at the limit ω->∞, which gives Rᵢ Rₑ/(Rᵢ+Rₑ)

c) (i) the relaxation frequency fᵣ at which there is a change in the impedance
(ii) the impedance Zᵣ of the transfer function at that change frequency
(iii) the gradient α of the change which gives the relaxation factor.

The impedance properties may be used to determine further impedance properties using the model.

For example, if both Rₑ and Rᵢ Rₑ/(Rᵢ+Rₑ) are known then Rᵢ can be determined.

The impedance Zᵣ of the transfer function at the change (dispersion) frequency fᵣ, is where the capacitor dominates the transfer characteristic as with each small increases in frequency it conducts significantly better reducing the impedance. The impedance Zᵣ at the change (dispersion) frequency fᵣ, can be modelled as 1/(j.2 π fᵣ.C). Therefore C can be determined as 1/(j.2π fᵣ. Zᵣ).

Variations of the individual impedance properties (Rₑ, Rᵢ, f₁, Z₁, α, C) with location may be used to locate a morphology, such as a micro bio-channel under skin, a meridian line or an acupuncture point.

However, the amount of variation of the individual impedance properties may be insufficient to enable accurate differential analysis of the structure by location. For example, the amount of variation of cell membrane capacitance (C) or relaxation frequency (fᵣ) may be insufficient to be readily detectable, for example based on those individual impedance properties.

In embodiments of the invention, selected predetermined impedance properties are ‘constructively’ combined to provide a parametric impedance value for the object. Constructive combination of the impedance properties in this way to provide parametric impedance value emphasises the variation of the individual electrical impedance properties. This enables the structure of the object to be more accurately analyzed.

Taking a simple example, if there is a 10% increase in one of the electrical impedance properties, such as cell membrane capacitance (C) from an initial value C₁ to 1.1C₁, and a 10% increase in another of the electrical impedance properties, such as relaxation frequency (fᵣ) from an initial value fᵣ₁ to 1.1fᵣ₁, these individual 10% increases may be insufficient to be readily detectable, for example discernible in measurements of these individual electrical impedance properties. However, combination of these individual electrical impedance properties by multiplication to provide a parametric impedance value will result in a larger increase of 21% (1.21 fᵣ₁C₁), which is more readily detectable.

An impedance property may have a positive, neutral or negative correlation with a particular morphology. A positive correlation means it increases, although perhaps not significantly, when the morphology is present. A negative correlation means it decreases, although perhaps not significantly, when the morphology is present. A neutral correlation means it does not change when the morphology is present. An impedance property with a positive correlation can be converted to one with a negative correlation (and vice versa) by taking the inverse.

Constructive combination of impedance properties for detecting a particular morphology means that impedance properties that are correlated in the same sense for that morphology are combined by multiplication (or weighted addition) to create the parametric impedance value and impedance properties that are correlated in the opposite sense for that morphology are combined by division (or weighted subtraction).

Any of the determined impedance properties may be constructively combined in any desired manner to provide a parametric impedance value that has a greater sensitivity to morphological changes that any of the constituent impedance properties.

Non-limiting examples of the combinations of impedance properties at the limiting level described in FIG. 3:

Combinational Parametric Measurements

(Combined Intra/Extra/Membrane Impedance/Conductivity)

a) Membrane impedance/conductivity and related quantities:

Membrane impedance: Zm=1/2π*fr*C

Membrane conductivity: σm=2π*fr*C

b) Combined Intracellular impedance/conductivity:

Product or Quotient: Ri*Zm or Ri/Zm or Ri/C or RiC
Or: σi*σm

Difference/Normalised Difference:
a*Ri−b*Zm
Or: c*σi−d*σm

Where coefficients a, b, c and d are constant (−∞−+∞) to be used for match the quantity to be used;

Differential/Normalised Differential:
(a*Ri−b*Zm)/Zm
Or: (a*Ri−b*Zm)/Ri
Alternatively: (c*σi−d*σm)/σm
Or: (c*σi−d*σm)/σi

Where coefficients a, b, c and d are constant (−∞−+∞) to be used for match the quantity to be used;

Intra-cellular time constant: Ri*C
Or: Intra-cellular frequency constant 1/Ri*C

c) Combined extra-cellular impedance/conductivity:

Product or Quotient: Re*Zm or Re/Zm or Re/C or ReC
Or: σx*σm

Difference/Normalised Difference:
a*Re−b*Zm
Or: c*σx−d*σm

Where coefficients a, b, c and d are constant (−∞−+∞) to be used for match the quantity to be used;

Differential/Normalised Differential:
(a*Re−b*Zm)/Zm
Or: (a*Re−b*Zm)/Re
Alternatively: (c*σx−d*σm)/σm
Or: (c*σx−d*σm)/σx

Where coefficients a, b, c and d are constant (−∞−+∞) to be used for match the quantity to be used;

Extra-cellular time constant: Re*C
Or: Extra-cellular frequency constant 1/Re*C

d) Combined extra-to-intra cellular impedance/conductivity:

Product: Re*Ri
Or: σx*σi

Quotient: Re/Ri or Ri/Re

Difference/Normalised Difference:
a*Re−b*Ri
Or: c*σx−d*σi

Where coefficients a, b, c and d are constant (−∞−+∞) to be used for match the quantity to be used;

Differential/Normalised Differential:
(a*Re−b*Ri)/Ri
Or: (a*Re−b*Ri)/Re
Alternatively: (c*σx−d*σi/σi
Or: (c*σx−d*σi/σx

Where coefficients a, b, c and d are constant (−∞−+∞) to be used for match the quantity to be used;

e) Triple combination:

ReRiC or ReRi/Cor

(Ri/Re)C or (Ri/Re)/C or

(Ri*Re)Zm or (Ri*Re)/Zm or . . . .

Combinational integrated cellular parametric measurements with deviant dispersion characteristic (a)

a) “Deviant” membrane impedance/conductivity and related quantities:

“Deviant” membrane impedance:
α*Zm
Or: α/Zm

“Deviant” membrane conductivity:
α*σm
Or: α/σm

b) Combined “deviant” Intra-cellular impedance/conductivity:

Product: α*Ri*Zm
Or: σi*σm

Difference/Normalised Difference:
α*(a*Ri−b*Zm)
Or: α*(c*σi−d*σm)

Where coefficients a, b, c and d are constant (−∞−+∞) to be used for match the quantity to be used;

Differential/Normalised Differential:
α*(a*Ri−b*Zm)/Zm
Or: α*(a*Ri−b*Zm)/Ri
Alternatively: α*(c*σi−d*σm)/σm
Or: α*(c*σi−d*σm)/σi

Where coefficients a, b, c and d are constant (−∞−+∞) to be used for match the quantity to be used;

Intra-cellular time constant: α*(Ri*C)
Or: Infra-cellular frequency constant α*(1/Ri*C)

c) Combined extra-cellular impedance/conductivity:

Product: α*Re*Zm

Or: α*σx*σm

Difference/Normalised Difference:
α*(a*Re−b*Zm)
Or: α*(c*σx−d*σm)

Where coefficients a, b, c and d are constant (−∞−+∞) to be used for match the quantity to be used;

Differential/Normalised Differential:
α*(a*Re−b*Zm)/Zm
Or: α*(a*Re−b*Zm)/Re
Alternatively: α*(c*σx−d*σm)/σm
Or: α*(c*σx−d*σm)/σx

Where coefficients a, b, c and d are constant (−∞−+∞) to be used for match the quantity to be used;

Extra-cellular time constant: α*Re*C
Or: Extra-cellular frequency constant α*(1/Re*C)

d) Combined extra-to-intra cellular impedance/conductivity:

Product: α*Re*Ri
Or: α*σx*σi

Difference/Normalised Difference:
α*(a*Re−b*Ri)
Or: α*(c*σx−d*σi)

Where coefficients a, b, c and d are constant (−∞−+∞) to be used for match the quantity to be used;

Differential/Normalised Differential:
α*(a*Re−b*Ri)/Ri
Or: α*(a*Re−b*Ri)/Re
Alternatively: α*(c*σx−d*σi)/σi
Or: α*(c*σx−d*σi)/σx

Where coefficients a, b, c and d are constant (−∞−+∞) to be used for match the quantity to be used;

e) Triple combination:

α*ReRiC or α*ReRi/C or

α*(Ri/Re)C or α*(Ri/Re)/C or

α*(Ri*Re)Zm or α*(Ri*Re)/Zm or . . . .

Combinational integrated cellular parametric measurements with dispersion frequency Fr. Replace α in a) to e) directly above with Fr.

Combinational integrated cellular parametric measurements with dispersion frequency Fr and dispersion characteristic (α). Replace α in a) to e) directly above with α*Fr.

A suitable impedance emphasising algorithm may be implemented by the computer 18 to select the optimum electrical impedance properties for combination and their manner of combination to maximise the variation of the resultant parametric impedance values.

After the parametric impedance value has been obtained for the object at one location, it is obtained at other locations. The method then identifies a location by a variation of the at least one parametric impedance value for the object. For example, a micro bio-channel under skin, a meridian or acupuncture point is identified by a predetermined spatial variation of one or more parametric impedance value for the object.

FIG. 4 illustrates a more generic model of the object under analysis. In the illustrated embodiment, the equivalent electrical impedance circuit 30 comprises an inclusion portion 31 in parallel with an inter-inclusion portion 33. The inclusion portion 31 has impedance Z1 and impedance Z2 in series. The impedance Z1 may be associated with the inclusion boundary (may be representative of the membrane related components of a group of cells) and the impedance Z2 may associated with the interior of the inclusion (may be representative of the intra-cellular related components of a group of cells). The inter-inclusion portion 33 has impedance Z3. The impedance Z3 is associated with the structure outside the inclusion (may be representative of extracellular components of a group of cells). The impedance Z3 is connected in parallel with the series connected impedance Z1 and Z2.

The impedance transfer function for this circuit is:

$$Z ( ω ) = \frac{Z 1 \cdot Z 2 \cdot Z 3}{Z 1 \cdot Z 2 + Z 1 \cdot Z 3 + Z 2 \cdot Z 3}$$

Non-limiting examples of the combinations of impedance properties at the level described in FIG. 4:

Combinational Parametric Measurements

a) Inclusion boundary impedance/conductivity and related quantities:

Inclusion boundary impedance: Zm=1/2π*fr*Z2 Inclusion boundary conductivity: σm=2π*Fr*Z2

b) Combined Intra-inclusion impedance/conductivity:

Product: Z1*Zm
Or: σ1*σm

Difference/Normalised Difference:
a*Z1−b*Zm
Or: c*σ1−d*σm

Where coefficients a, b, c and d are constant (−∞−+∞) to be used for match the quantity to be used;

Differential/Normalised Differential:
(a*Z1−b*Zm)/Zm
Or: (a*Z1−b*Zm)/Z1
Alternatively: (c*σ1−d*σm)/σm
Or: (c*σ1−d*σm)/σ1

Where coefficients a, b, c and d are constant (−∞−+∞) to be used for match the quantity to be used;

Intra-inclusion time constant: Z1*Z2
Or: Intra-inclusion frequency constant 1/Z1*Z2

c) Combined inter-inclusion impedance/conductivity:

Product: Z3*Zm
Or: σ3*σm

Difference/Normalised Difference:
a*Z3−b*Zm
Or: c*σ3−d*σm

Where coefficients a, b, c and d are constant (−∞−+∞) to be used for match the quantity to be used;

d) Combined inter-to-intra inclusion impedance/conductivity:

Product: Re*Ri
Or: σx*σi

Difference/Normalised Difference:
a*Re−b*Ri
Or: c*σx−d*σi

Where coefficients a, b, c and d are constant (−∞−+∞) to be used for match the quantity to be used;

Differential/Normalised Differential:
(a*Re−b*Ri/Ri
Or: (a*Re−b*Ri)/Re
Alternatively: (c*σx−d*σi/σi
Or: (c*σx−d*σi/σx

Where coefficients a, b, c and d are constant (−∞−+∞) to be used for match the quantity to be used;

Differential/Normalised Differential:
(a*Z3−b*Zm)/Zm
Or: (a*Z3−b*Zm)/Z3
Alternatively: (c*σ3−d*σm)/σm
Or: (c*σ3−d*σm)/σ3

Where coefficients a, b, c and d are constant (−∞−+∞) to be used for match the quantity to be used;

Inter-inclusion time constant: Z3*Z2
Or: Inter-inclusion frequency constant 1/Z3*Z2

Combinational integrated parametric measurements with deviant dispersion characteristic (α)

a) “Deviant” inclusion boundary impedance/conductivity and related quantities:

“Deviant” inclusion boundary impedance:
α*Zm
Or: α/Zm

“Deviant” inclusion boundary conductivity:
α*σm
Or: α/σm

b) Combined “deviant” Infra-inclusion impedance/conductivity:

Product: α*Z1*Zm
Or: σ1*σm

Difference/Normalised Difference:
α*(a*Z1−b*Zm)
Or: α*(c*σ1−d*σm)

Where coefficients a, b, c and d are constant (−∞−+∞) to be used for match the quantity to be used;

Differential/Normalised Differential:
α*(a*Z1−b*Zm)/Zm
Or: α*(a*Z1−b*Zm)/Z1
Alternatively: α*(c*σ1−d*σm)/σm
Or: a*(c*σ1−d*σm)/σ1

Where coefficients a, b, c and d are constant (−∞−+∞) to be used for match the quantity to be used;

Intra-inclusion time constant: α*(Z1*Z2)
Or: Intra-inclusion frequency constant α*(1/Z1*Z2)

c) Combined inter-inclusion impedance/conductivity:

Product: α*Z3*Zm
Or: σ3*σm

Difference/Normalised Difference:
α*(a*Z3−b*Zm)
Or: σ*(c*σ3−d*σm)

Where coefficients a, b, c and d are constant (−∞−+∞) to be used for match the quantity to be used;

Differential/Normalised Differential:
α*(a*Z3−b*Zm)/Zm
Or: α*(a*Z3−b*Zm)/Z3
Alternatively: α*(c*σ3−d*σm)/σm
Or: α*(c*σ3−d*σm)/σ3

Where coefficients a, b, c and d are constant (−∞−+∞) to be used for match the quantity to be used;

Inter-inclusion time constant: α*Z3*Z2
Or: Inter-inclusion frequency constant α*(1/Z3*Z2)

d) Combined inter-to-intra inclusion impedance/conductivity:

Product: α*Re*Ri
Or: α*αx*σi

Difference/Normalised Difference:
α*(a*Re−b*Ri)
Or: α*(c*σx−d*σi)

Where coefficients a, b, c and d are constant (−∞−+∞) to be used for match the quantity to be used;

Differential/Normalised Differential:
α*(a*Re−b*Ri)/Ri
Or: α*(a*Re−b*Ri)/Re
Alternatively: α*(c*σx−d*σi)/σi
Or: α*(c*σx−d*σi)/σx

Where coefficients a, b, c and d are constant (−∞−+∞) to be used for match the quantity to be used;

This model is a fractal model as previously described in U.S. Pat. No. 6,856,824. Each of the impedances Z1, Z2, Z3 may be represented using either the circuit 30 or at the limiting level where Z1 is equivalent to Ri, Z2 is equivalent to C and Z3 in equivalent to Re. The term ‘fractal’ is used to express the fact that at whatever level of dimension one looks at the structure the model is the same.

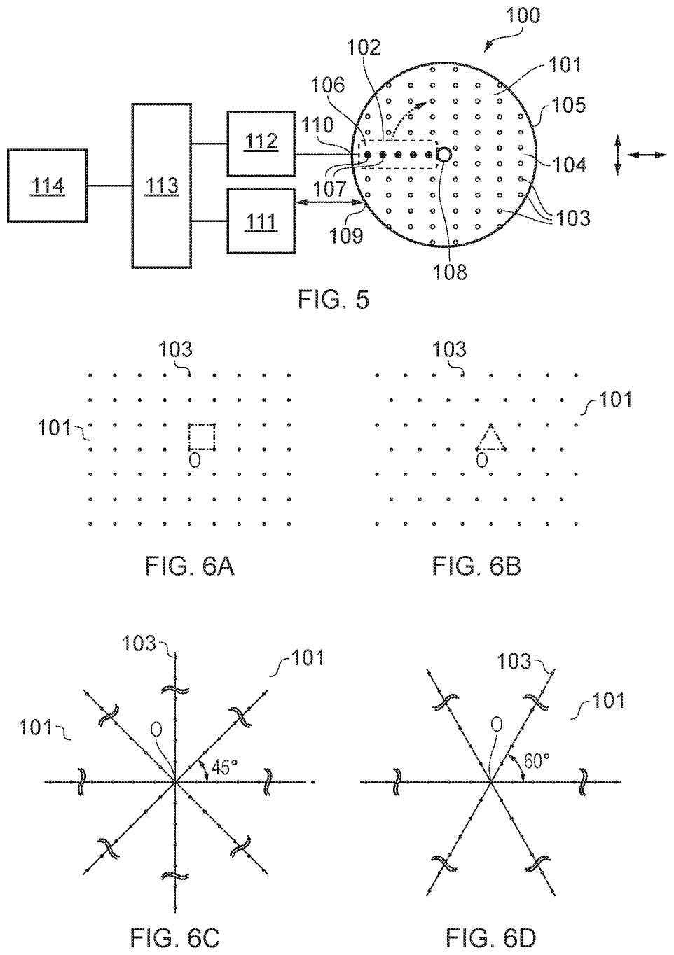

Referring to FIG. 5, there is illustrated an apparatus 10 for detecting signals characteristic of body tissue comprising an electrode array 101 and an ultrasound probe 102.

The electrode array 101 comprises a plurality of electrodes 103 disposed on a face 104 of an electrode plate 105. In use, the body tissue (not illustrated) is adjacent the electrode plate 105, adjacent to a face 104 of the electrode plate 105, either in contact with, or spaced apart from, the face 104. The electrodes 103 are able to apply a first electrical signal to the body tissue during electrical impedance measurements on the body tissue. The electrodes 103 are electrically coupled to a first controller 111 for transmitting the first electrical signal to the electrodes 103 for applying to the body tissue and for receiving a first output signal from electrodes 103, which first output signal depends on electrical response signals, characteristic of the body tissue, received at the electrodes 103.

The ultrasound probe 102 comprises a plurality of ultrasound transducers 107 disposed on a face 106 of the ultrasound probe 102. The ultrasound transducers 107 are able to apply a first ultrasound signal to the body tissue during ultrasound examination on the body tissue. The ultrasound transducers 107 are electrically coupled to a second controller 112 for providing a second input signal, generally in the form of electrical pulses, to the ultrasound transducers 107 that cause the ultrasound transducers 107 to apply the first ultrasound signal to the body tissue, and for receiving a second output signal from the ultrasound transducers 107, which second output signal depends on ultrasound response signals, characteristic of the body tissue, received at the ultrasound transducers 107.

The face 106 of the ultrasound probe 102 on which the ultrasound transducers 107 are disposed is adjacent to the electrode plate 105 and on the opposite side of the electrode plate 105 to the face 104 of the electrode plate 105 on which the electrodes 103 are disposed. Therefore, if the electrode plate 105 is placed horizontally with the face 104 of the electrode plate 105 on which the electrodes 103 are disposed upwards, then the ultrasound probe 102 is beneath the electrode plate 105 with the face 106 of the ultrasound probe 102 on which the ultrasound transducers 107 are disposed also upwards. Therefore, the ultrasound transducers 107 are arranged in a plane substantially parallel to the electrode plate 105. This enables the electrical signals and the ultrasound signals to be applied to the body tissue in directions that are substantially parallel to each other.

Movement of the electrode plate 105 enables the first electrical signals to be applied, and the electrical response signals to be detected, at different locations over a region of the body tissue larger than the area of the electrode plate 105 over which the electrodes 103 are deployed. The system 13 may cause translation of the electrode plate 105 and ultrasound probe 102 relative to the subject as described previously with reference to FIG. 1.

In this example, the ultrasound probe 102 also rotates relative to a subject. The ultrasound probe 102 and the electrode plate 105 are mechanically coupled, whereby the ultrasound probe 102 is rotatable about an axis 108 substantially perpendicular to the electrode plate 105. For a region of the body tissue of a given size, fewer electrodes 103 may be deployed, which can reduce the complexity of electrical connections. Rotation of the electrode plate 105 also enables electrical measurements with a fine resolution, using incremental positions of the electrodes 103 more closely spaced than the physical spacing on the electrodes 103.

The electrodes 103 are coupled to a first port 109, and the ultrasound transducers 107 are coupled to a second port 110. The first port 109 is bidirectional, for conveying signals to and from the electrodes 103. The second port 110 is also bidirectional, for conveying signals to and from the ultrasound transducers 107. For clarity, connections between the first port 109 and the electrodes 103, and between the second port 110 and the ultrasound transducers 107, are not illustrated in FIG. 1. These connections may, for example, be located on the face of the electrode plate 105 opposite to the face 104, or may be internal to the electrode plate 105.

There is a first controller 111 coupled to the first port 109. The first controller 111 generates the first input signal which is delivered via the first port 109 to one or more of the electrodes 103 where, in response to the first input signal, the first electrical signal is transmitted to the body tissue. The first electrical signal passes through the body tissue and is received at other of the electrodes 103. These received signals are termed electrical response signals in this specification and the accompanying claims. The first output signal, dependent on the electrical response signals is delivered to the first controller 111 via the first port 109.

There is a second controller 112 coupled to the second port 110. The second controller 112 generates the second input signal which is delivered via the second port 110 to the ultrasound transducers 107. The second input signal may be, for example an electrical signal or optical signal. The ultrasound transducers 107 convert the second input signal to the first ultrasound signal which is transmitted to the body tissue. The first ultrasound signal is reflected in the body tissue. These reflections are termed ultrasound response signals in this specification and the accompanying claims. The ultrasound response signals are detected by the ultrasound transducers 107, which convert the ultrasound response signals to the second output signal which is delivered to the second controller 112 via the second port 110.

The first and second controllers 111, 112 are coupled to a computer 18. The computer 18 generates electrical impedance data based on the first output signal, and ultrasound data based on the second output signal. The ultrasound data and the electrical impedance data are characteristic of the body tissue.

Alternatively, the computer 18 may combine the ultrasound data and the electrical impedance data, and the display 114 (optional) may display an image representative of the combined ultrasound data and electrical impedance data. By this means, the electrical impedance data and the ultrasound data may be combined to provide an enhanced image, which can assist detection and location of features of the body tissue such as micro bio-channels under skin, meridian lines and acupuncture points. Features of the body tissue that may not be apparent from solely the electrical impedance data or the ultrasound data may become apparent after the combination of the electrical impedance data and the ultrasound data. The images may be two or three dimensional.

Although the electrode plate 105 illustrated in FIG. 5 is flat, this is not an essential feature of the invention, and the electrode plate 105, or at least the face 104, may be non-flat. For example, the face 104 may be profiled in a similar shape to the body tissue. This enables distortion of the shape of the body tissue to be reduced or avoided. The face 106 of the ultrasound probe 102, and the arrangement of ultrasound transducers 107 may be profiled to complement the shape of the adjacent electrode plate 105.

By employing shapes which are complementary to the shape of the body tissue, the length of the signal path between the electrode plate 105 and the body tissue, and between the ultrasound transducers 107 and the body tissue, may be reduced, resulting in improved sensitivity of the apparatus in detecting the response signals.

In the embodiments illustrated in FIG. 4 the electrode plate 105 is circular. This is not an essential feature of the invention, and other shapes may be used.

The signals delivered via the first port 109 and the second port 110 may be electric currents or voltages, or may be optical signals. Also, they may be analogue or digital signals. Where optical signals are used, conversion between optical and electrical signals may be performed by the ultrasound transducers 107, by the electrodes 103 and by the first and second controllers 111, 112. Digital to analogue conversion, and analogue to digital conversion, may be performed by the ultrasound transducers 107, by the electrodes 103 and by the first and second controllers 111, 112. The ultrasound transducers 107, the electrodes 103 and the first and second controllers 111, 112 may include signal processing, for example amplification and filtering. The first controller 111 may be integral with the electrode plate 105 and the second controller 112 may be integral with the ultrasound probe 102, in which case either or both of the first and second ports 109, 110 may be internal to the electrode plate 105 or ultrasound probe 102 respectively. Alternatively the first controller 111 may be spaced apart from the electrode plate 105 by means of cables, and/or the second controller 112 may be spaced apart from the ultrasound probe 102 by means of cables.

The first controller 111 and the second controller 112 may be coupled, and indeed may be a common controller. This enables the generation of the first and second signals to be synchronised. For example, the relative timing and/or the magnitude of the first and second signals may be controlled.

The apparatus 10 may comprise a spacing member for spacing an object under evaluation from the electrode carrier plate 105. When the electrode carrier plate 105 rotates, the spacing member and the object do not rotate. In this way, the object is shielded from rotational forces from the rotating carrier plate 105, and discomfort to a patient can be reduced or eliminated. The rotatable carrier plate 105 may have an ultrasound window for the attached ultrasound probe 106. The window may be an aperture through the plate 105. The spacing membrane 800 may be made from an ultrasonically transparent membrane such as, for example, a polymer e.g. a high electrical impedance polymer. In this example, each electrode may have an aperture through the spacing member.

FIGS. 6A, 6B, 6C, 6D discloses examples of an electrode array 101 of electrodes 103. These electrode arrays 101 are rotatable and have rotation symmetry. A spacing member is used for spacing an object under evaluation from the rotating electrode array 101. The carrier plate 105 is electrically non-conductive and which may be made, for example, of a plastic material. Electrodes 103 are deployed across a flat surface of the electrode carrier plate 105 and are preferably recessed in the electrode carrier plate 105 so that they do not make physical contact with an object placed on the electrode carrier plate 105. Each electrode is denoted by a dot.

The electrodes are arranged on an electrode carrier plate 105 in an arrangement comprising a unit of repetition that repeats over the electrode carrier plate 105 and that has an angle of rotational symmetry less than or equal to 90°. Electrodes are deployed in such a manner enable measurement of electrical impedance to be made using a pattern of electrodes rotated through successive positions by a rotational displacement which is less than 90°.

In FIG. 6A, the electrodes 103 are arranged equidistant in a square matrix, such that the electrodes are located at corners of squares arranged in a continuum. The electrodes are arranged at one or more corners of each square of a tessellation of squares. Such an arrangement enables a rotational symmetry which is a multiple of 90°. In such an arrangement, each electrode, except those adjacent the boundary of the arrangement, has four nearest neighbour electrodes which are arranged in a square. A more dense matrix could alternatively be provided by subdividing each square into four smaller squares.

The electrodes 103 are in an X column by N row array where X=1, 2, 3, . . . N; Y=1, 2 . . . M; for example 3 by 16 array, 3 by 24 array; 5 by 16 array, 5 by 24 array, the gaps between electrodes can be any possible value, such as 0.01 mm, 0.02 mm, 0.05 mm, 0.1 mm, 0.5 mm, 1 mm etc

In FIG. 6B, the electrodes 103 are arranged equidistant in a triangular matrix, such that the electrodes are located at corners of equilateral triangles arranged in a continuum. The electrodes are arranged at one or more corners of each triangle of a tessellation of triangles. The triangles can be equilateral triangles. Furthermore, the triangles can be of equal size. Such an arrangement enables a rotational symmetry which is a multiple of 60°. In such an arrangement, each electrode, except those adjacent the boundary of the arrangement, has six nearest neighbour electrodes which are arranged in a hexagon. A more dense triangular matrix could alternatively be provided by subdividing each equilateral triangle into four smaller equilateral triangles.

The electrodes 103 are in a half-occupied X column by N row array X=1, 2, 3, . . . N; Y=1, 2 . . . M; for example 3 by 16 array, 3 by 24 array; 5 by 16 array, 5 by 24 array, the gaps between electrodes can be any possible value, such as 0.01 mm, 0.02 mm, 0.05 mm, 0.1 mm, 0.5 mm, 1 mm etc

In FIGS. 6C and 6D, the electrodes 103 are arranged on five or more radially extending lines passing through a common point O. In two dimensions, N radial lines have a regular separation of 360°/N and the gaps between electrodes 103 along the radial lines can be any possible value, such as 0.01 mm, 0.02 mm, 0.05 mm, 0.1 mm, 0.5 mm, 1 mm etc

The diameter of each electrode could be 1-5 um, but is not limited to this range. The central distance between closest electrodes could be in between 0.01-1 mm for example.

The electrodes 103 may be configured to measure up to 5-10 mm under skin, normally 2-4 mm at 1-2 mm resolution.

FIG. 7 illustrates an example of an apparatus 10 which may or may not use ultrasound measurement in addition to electrical impedance measurement. An electrode carrier plate 105 can be moved laterally 200 relative to an object (subject). The movement may be caused by the system 13 (not illustrated) which may, for example, comprise servo motors. The apparatus 10 moves the array of electrodes 103 and takes measurements at each location.

The apparatus:

(i) obtains electrical impedance data for the object at different locations;

(ii) analyses the obtained electrical impedance data using a transfer function of an assumed electrical model to determine a variation of a plurality of electrical impedance properties for the object with location; and

(iii) identifies a location identified by a variation of one or more of the plurality of electrical impedance properties.

Having identified the location of a variation of one or more of the plurality of electrical impedance properties the apparatus 10 moves the carrier plate 105 of the electrodes 103 so that an aperture 202 in the middle of the carrier plate 105 is positioned over the location which corresponds to a micro bio-channel under skin, a meridian or acupuncture point. This allows an acupuncturist to identify the acupuncture point within the aperture 202.

The apparatus 10 may be configured as a strap or band that is moved along a limb. The lateral movement is then across the limb.

The electrical impedance data for the object may be obtained over a range of frequencies at each one of multiple different locations;

Parametric impedance value for the object that vary with location may be formed by constructively combining selected ones of the determined plurality of electrical impedance properties and the location of interest may be identified by a variation of the at least one parametric impedance value for the object.

The ultrasonic sensors may be located within the aperture 202.

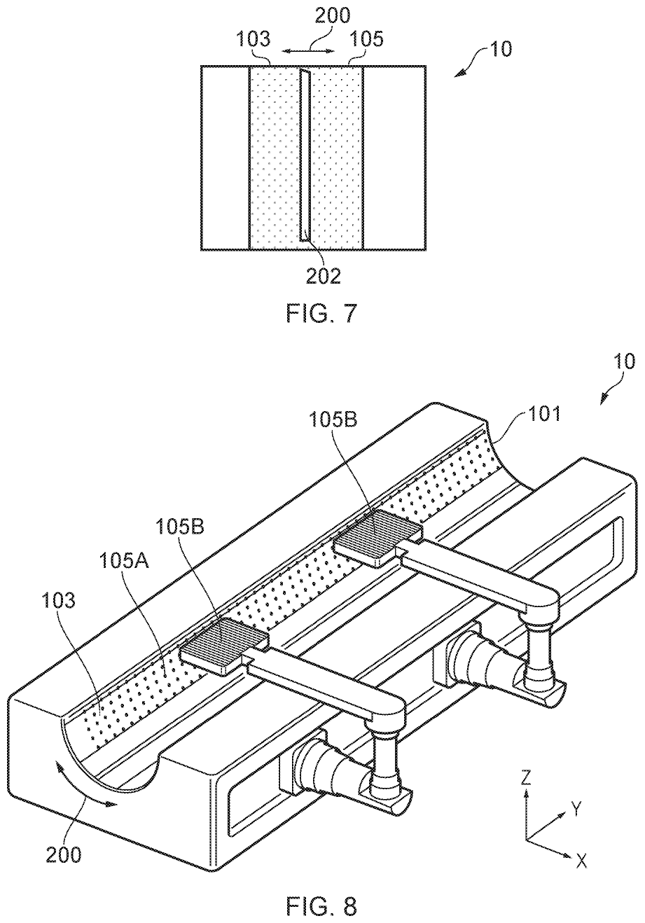

FIG. 8 illustrates an example of an apparatus 10 which may or may not use ultrasound measurement in addition to electrical impedance measurement. An electrode carrier plate 105 is divided into two portions 105A and 105B.

The portion 105A is an array 101 of electrodes 103. This array may be fixed or moveable. It may for example be moved laterally 200 relative to an object (subject). The movement may be caused by the system 13 (not illustrated) which may, for example, comprise servo motors.

The portion 105B is an array 101 of electrodes 103. This array may be moveable and operates a reading head 210. In the illustrated example there are two reading heads 210. It may for example be moved laterally and/or longitudinally relative to an object (subject). The movement may be caused by the system 13 (not illustrated) which may, for example, comprise servo motors.

The apparatus 10 moves the array of electrodes 103 and takes measurements at each location.

The apparatus 10:

(i) obtains electrical impedance data for the object at different locations;

(ii) analyses the obtained electrical impedance data using a transfer function of an assumed electrical model to determine a variation of a plurality of electrical impedance properties for the object with location; and

(iii) identifies a location identified by a variation of one or more of the plurality of electrical impedance properties.

Having identified the location of a variation of one or more of the plurality of electrical impedance properties the apparatus 10 moves the reading head(s) 210 to identify the location.

The electrical impedance data for the object may be obtained over a range of frequencies at each one of multiple different locations;

Parametric impedance value for the object that vary with location may be formed by constructively combining selected ones of the determined plurality of electrical impedance properties and the location of interest may be identified by a variation of the at least one parametric impedance value for the object.

In the foregoing examples, a temperature control sub-system may be used to provide constant or differential heating/cooling of the subject, for example, via the electrode plate 105 or independently. The measurements at the different locations may therefore be made under controlled fixed or variable temperature conditions.

In the foregoing examples, the measurements at the different locations may be made without acupuncture or may be made during acupuncture.

By applying time varying parameters such as temperature changes and absence/presence of acupuncture, it is possible to determine time-variation of the electrical impedance properties. This may allow determination of parameters, such as speed, transmission direction and sense of transmission along the meridian line during the acupuncture stimulation. The dynamic measurement detects the interstitial fluid flow in channels from the changes of data when acupuncture is given. The device can also tell the difference between meridian channel and blood vessel according to characteristic frequency of 1-3 MHz for red cells.

A database may be used to compare the measured the electrical impedance properties of a subject with the data under different gender, age, normal and abnormal groups.

Although embodiments of the present invention have been described in the preceding paragraphs with reference to various non-limiting examples, it should be appreciated that modifications to the examples given can be made without departing from the scope of the invention as claimed.

Whilst endeavoring in the foregoing specification to draw attention to those features of the invention believed to be of particular importance it should be understood that the Applicant claims protection in respect of any patentable feature or combination of features hereinbefore referred to and/or shown in the accompanying drawings.

1. A method for locating a variation in one or more electrical impedance properties of an object, the method comprising the steps of:
(i) obtaining electrical impedance data for the object at different locations;
(ii) analysing the obtained electrical impedance data using a transfer function of an assumed electrical model to determine a variation of a plurality of electrical impedance properties for the object with location; and
(iii) identifying a location identified by a variation of one or more of the plurality of electrical impedance properties.

2. A method as claimed in claim 1, comprising using an electrode array for performing electrical impedance detection to obtain the electrical impedance data for the object by applying a first electrical signal to the object, receiving an electrical response signal characteristic of the object, and providing a first output signal representative of the electrical response signal.

3. (canceled)

4. A method as claimed in claim 2, comprising physically moving at least a part or a whole of the electrode array laterally across a human limb to perform electrical impedance detection at different locations.

5. A method as claimed in claim 2, comprising physically moving at least a part or a whole of the electrode array so that an aperture in a carrier plate for the electrode array is positioned over the location which corresponds to the identified location, wherein ultrasonic sensors are located within the aperture.

6. (canceled)

7. A method as claimed in claim 2, wherein the electrode array is divided into two portions configured for movement relative to each other, the method comprising moving one of the portions of the electrode array to identify to a user the identified location.

8. (canceled)

9. A method as claimed in claim 1, wherein the identified location locates a human morphology and/or a micro bio-channel under skin and/or a sub-cutaneous micro-volume of low hydraulic resistance and/or a meridian and/or an acupuncture point.

10. A method as claimed in claim 9, further comprising providing heating/cooling.

11. A method as claimed in claim 1, wherein the electrode array is mounted on a movable element of the apparatus that is configured, in use, to move with respect to the body tissue of the object, wherein the movable element has a window.

12. (canceled)

13. An apparatus as claimed in claim 11, wherein the window is an aperture in the movable element.

14. An apparatus as claimed in claim 11, wherein electrodes of the electrode array are not present over the window.

15. A method as claimed in claim 13 comprising using a spacing member between the movable element and the subject, wherein the spacing member is ultrasonically transparent and non-conductive.

16. (canceled)

17. A method as claimed in claim 15, wherein apertures of the spacing member are configured for alignment, in use, with electrodes of the electrode array, wherein the apertures of the spacing member are arranged in a configuration with symmetry and wherein the electrodes of the electrode array are arranged in a configuration with corresponding symmetry.

18. (canceled)

19. A method as claimed in claim 1, comprising using an ultrasound transducer to perform ultrasound detection by applying a first ultrasound signal to the object, receiving an ultrasound response signal characteristic of the object, and providing a second output signal representative of the ultrasound response signal, the method comprising moving at least a part or a whole of the ultrasound probe relative to the object.

20. (canceled)

21. A method according to claim 1, wherein the electrical model assumes first and second serially connected impedances connected in parallel with a third impedance or
wherein the electrical model assumes a capacitor and a serially connected resistor, which are connected in parallel with another resistor, wherein the electrical impedance properties are selected from the group comprising:
an impedance at a lower frequency limit,
an impedance at an upper frequency limit,
a relaxation frequency fr at which there is a change in the impedance,
an impedance at that relaxation frequency, and
the impedance gradient at that relaxation frequency.

22. (canceled)

23. (canceled)

24. A method as claimed in claim 1 wherein obtaining electrical impedance data for the object at different locations comprises:
obtaining electrical impedance data for the object at different locations over a range of frequencies wherein the frequency range is between 0 and 100 MHz.

25. (canceled)

26. An apparatus for locating a variation in one or more electrical impedance properties of a human subject comprising:
(i) means for obtaining electrical impedance data for the human subject at different locations on the human body;
(ii) means for analysing the obtained electrical impedance data using a transfer function of an assumed electrical model to determine a variation of a plurality of electrical impedance properties for the object with location; and
(iii) means for identifying a location identified by a variation of one or more of the plurality of electrical impedance properties.

27. A system for locating a variation in one or more electrical impedance properties of an object, the system comprising:
means for obtaining electrical impedance data for the object at different locations;
means for analysing the obtained electrical impedance data using a transfer function of an assumed electrical model to determine a variation of a plurality of electrical impedance properties for the object with location;
means for identifying a location identified by a variation of one or more of the plurality of electrical impedance properties.

28. An apparatus comprising a processor and a memory storing a computer program for locating a variation in one or more electrical impedance properties of an object, wherein the processor, memory and computer program enable the apparatus to:
analyse electrical impedance data using a transfer function of an assumed electrical model to determine a variation of a plurality of electrical impedance properties for the object with location; and
identify a location identified by a variation of one or more of the plurality of electrical impedance properties.

29. (canceled)

30. The apparatus as claimed in claim 28, comprising an electrode array configured to perform electrical impedance detection to obtain the electrical impedance data for the object by applying a first electrical signal to the object, receive an electrical response signal characteristic of the object, and provide a first output signal representative of the electrical response signal and comprising the electrode array.

31. An apparatus as claimed in claim 30, comprising one or more motors configured to physically move one or more electrode carrier plates carrying the electrode array, laterally across a human limb to perform electrical impedance detection at different locations and wherein the carrier plate comprises an aperture for accessing an acupuncture point and the identified location corresponds to then acupuncture point and comprising ultrasonic sensors located within the aperture.
